(19) United States
(12) Patent Application Publication
(10) Pub. No.: US 2024/0081909 A1
(43) Pub. Date: Mar. 14, 2024

(54) SUPPRESSION OF DEVITRIFICATION IN SURGICAL FIBER OPTICS

(71) Applicant: Cyclone Biosciences, LLC, Phoenix, AZ (US)

(72) Inventor: Stephen E. Griffin, Peoria, AZ (US)

(73) Assignee: Cyclone Biosciences, LLC, Phoenix, AZ (US)

(21) Appl. No.: 18/242,960

(22) Filed: Sep. 6, 2023

Related U.S. Application Data
(63) Continuation-in-part of application No. 18/228,983, filed on Aug. 1, 2023.
(60) Provisional application No. 63/414,974, filed on Oct. 11, 2022.
(60) Provisional application No. 63/405,984, filed on Sep. 13, 2022.

(51) Int. Cl. A61B 18/24; G02B 6/34

(52) U.S. Cl. CPC A61B 18/24; G02B 6/34; A61B 2018/2244

(57) ABSTRACT

A tubular optically-transparent device structured as a terminating cap for an optical fiber is configured to deliver laser radiation from the optical fiber substantially radially (generally—transversely to the axis of the cap) while at the same time spatially-redistributing the radiant intensity at a point of interaction of laser radiation with a wall of the cap to reduce material damage of the material of the cap thereby reducing tissue adhesion during surgery performed with the use of the device including such optical fiber and cap. A method for operating the same.

### CROSS-REFERENCE TO RELATED APPLICATIONS

The present US patent application claims priority from and benefit of the US Provisional Patent Application No. 63,414,974 filed on Oct. 11, 2022. The present US patent application is also a continuation-in-part from the U.S. patent application Ser. No. 18/228,983 filed on Aug. 1, 2023, which claims priority from the U.S. Provisional Patent Application No. 63/405,984 filed on Sep. 13, 2022. The disclosure of each of the above-identified patent document(s) is incorporated by reference herein.

### TECHNICAL FIELD

This invention relates to systems and methods configured to alter or modify a profile of light output from fiber optics that are useful in both optimization of treatment of various intracorporeal disease states (for example, a vaporization of benign prostatic hyperplastic tissues) with intense light such as laser light and in forestalling the onset (and slowing the progression) of devitrification damage the used fiber optic suffers in treating such diseases.

### RELATED ART

Optical fiber contraptions configured to produce optical output/emissions directed laterally are utilized in a variety of light-based surgical procedures. The examples of such procedures include vaporization of benign excess prostate tissue in Lower Urinary Tract Syndrome (LUTS) in men, and treating laryngeal tumors, to name just a few. Utilization of such fiber optics configured to produce light output directed transversely to an axis of the used optical fiber has been proposed for various additional surgical interventions such as ablation, vaporization, liquefaction and/or coagulation of tissue (including atherosclerotic and vulnerable plaques, partial nephrectomy, and even liposuction). Discussion of optical fiber structures configured to produce radially-directed optical output is presented, for example, in U.S. patent application Ser. No. 17/224,354 filed on Apr. 7, 2021 and now published as US 2021/0330383, the disclosure of which is incorporated herein by reference.

Regardless of the specifics of a particular spatial profile of the light output from a piece of fiber optics—whether this profile corresponds to a standard axial output from a flat polished optical fiber facet or to a substantially 360° radial output directed substantially orthogonally to the longitudinal axis of the optical fiber—those skilled in the art of laser surgery (and even in materials processing) recognize that the typical Gaussian profile of radiant intensity across the light output from the fiber is far from being optimal, in that such Gaussian profile often facilitates overexposure and underexposure of the target within the illuminated area. A person of skill in the art will agree that the more spatially uniform the distribution of irradiance in the optical fiber output can be, the better optimum treatment can be accomplished, thereby reducing surgical time and complication resulting from overtreatment. Methodologies available for conditioning a laser output to produce a more of a “top hat” output profile in the free-beam realm (that is, in shaping the laser beam before it is launched into the surgical optical fiber) include diffractive lenses and diffusers such as those offered by HOLO/OR Limited, Israel, for example. Such “laser beam conditioning”, however, results in preloading a surgical optical fiber with higher-order spatial modes of light that render the fiber susceptible to burn-through in the region where the fiber is tightly bent (and, as is well known, tight bending of the fiber is typically required for accessing intracorporeal spaces). As such, it is necessary to preferentially excite predominantly low-order modes within a surgical optical fiber in order to safely deliver high energy to target tissues, but the so-limited coupling conditions produce outputs with semi-Gaussian and semi-Lambertian profiles that are undesirable in surgery.

The need remains to devise a mechanism that flattens fiber-optic light output profiles at or near the fiber output, makes it more “top hat” or more spatially uniform—and, in particular, a light output produced by the so-called side-fire surgical fiber optic contraptions.

### SUMMARY OF THE INVENTION

Embodiments of the invention provide an article of manufacture that includes an optical fiber termination cap. Such cap contains an optically-transparent tube (having a tube axis, an open end dimensioned to receive an optical fiber along a tube axis, and a closed end) and an internal optical element. The internal optical element has an output optical element surface transverse to the tube axis and is configured inside the tube (i) to receive light along the tube axis from the open end and, (ii) upon interaction of said light with the output optical element surface, to form a first beam of light having a first degree of spatial divergence and directed transversely to the tube axis along an output axis through a wall of the tube. Here, the wall of the tube contains a surface relief structure intersecting the output axis. Such surface relief structure is configured to transmit the first beam of light therethrough while changing the first degree of spatial divergence to a different second degree of spatial divergence. In at least one case, the surface relief structure may include one or more of a periodic surface corrugation pattern and an aperiodic surface corrugation pattern and/or be dimensioned to substantially circumscribe the tube around the tube axis and/or be formed at least in part on a substantially-planar portion of an outer surface of the wall of the tube and/or be dimensioned to define a lens element. Substantially in every implementation of the article of manufacture, at least a portion of the surface relief structure may be configured as an optical metasurface. In at least one specific implementation, the output optical element surface may be made substantially planar and/or the optical element may include an input optical element surface defining a surface of a lens to receive the light along the tube axis from the open end and be configured to form an intermediate beam of light that has the first degree of spatial divergence and that propagates towards the output optical element surface. (Optionally, in such specific case, and when the optical element includes an input optical element surface, such input optical element surface may be configured to direct the intermediate beam of light through a body of the optical element.) In one or more implementations, the article may additionally include a cannula connected to the optical fiber termination cap (and, in one specific implementation, there may be a cannula-mount segment of a fiber-control device affixed to the cannula and/or the open end may be dimensioned to receive an optical fiber along a tube axis and/or the article of manufacture may additionally include the optical fiber cooperated with the fiber control device and inserted into the optical fiber termination cap). Optionally, the article of manufacture may include a centering sleeve disposed about an output tip of the optical fiber. Substantially in every embodiment, the open end may be dimensioned to receive an optical fiber along a tube axis, while the article additionally includes an optical fiber inserted into the tube (here, in at least one case, an output facet of the optical fiber may be shaped to include a curved surface).

Embodiments of the invention additionally provide a method that includes a step of interacting light (that propagates axially within an optical termination cap from an open end of an optical termination cap towards a closed end of the optical termination cap) with an optical body that is located inside the optical termination cap and that has an optical body output surface transverse to an optical termination cap axis. (Here, such optical termination cap includes an optically-transparent tube having the open end, the closed end, and the optical termination cap axis.) The method additionally includes a step of—upon so interacting—forming a first beam of light having a first degree of spatial divergence and directed transversely to the optical termination cap axis and along an output axis through a wall of the tube; and a step of transforming the first beam of light into a second beam of light having a second degree of spatial divergence by transmitting the first beam of light through a surface relief structure on the wall of the tube (where the second degree of spatial divergence being different from the first degree of spatial divergence). Optionally, the open end may be dimensioned to receive an optical fiber along the optical termination cap axis, and the method may additionally include a step of outcoupling the light through an output facet of an optical fiber secured in the optical fiber termination cap. At least when the latter is the case, the method may be configured to satisfy one or more of the following conditions: (1) the step of outcoupling light includes outcoupling the light from an output facet of an optical fiber, and (2) the step of outcoupling light includes outcoupling the light from the output facet of the optical fiber (such output facet being a facet of the optical fiber taper), and (3) the step of outcoupling light includes outcoupling the light from the output face, while output facet is a facet of the optical fiber element that includes: (i) an optical fiber having a fiber core and a fiber cladding, and (ii) an optical termination element in contact with an input facet of the optical fiber, the optical termination element having a front surface, a termination core, and a termination cladding (here, a first ratio of a termination core diameter to a termination cladding diameter may be made substantially equal to a second ratio of a fiber core diameter to the fiber cladding diameter). In at least one implementation of the method, the output facet of the optical fiber may be structured to include a curved surface transverse to the optical termination cap axis, and/or the step of transforming the first beam may include transmitting light of the first beam through the surface relief structure that includes one or more of a periodic surface corrugation pattern and an aperiodic surface corrugation pattern and/or that is dimensioned to form a lens element. Substantially in every implementation of the method employing the step of transforming, such step may include transmitting the first beam through the surface relief structure that is dimensioned to substantially circumscribe the tube around the optical termination cap axis and/or that is formed at least in part on a substantially-planar portion of an outer surface of the wall of the tube and/or that includes an optical metasurface. Alternatively or in addition—and when the optical termination cap contains an optical fiber inserted into the cap through the open end and spatially secured with respect to the cap—the method may additionally include a step of operating a fiber control device cooperated with the optical fiber to spatially reposition the optical termination cap axis.

### BRIEF DESCRIPTION OF THE DRAWINGS

The invention will be more fully understood by referring to the following Detailed Description of Specific Embodiments in conjunction with the Drawings, of which:

FIG. 1 presents a side view of a portion of a conventionally-structured optical-fiber cap of related art damaged as a result of devitrification caused by laser light transmitted radially from inside the cap through the cap's wall to form the radially-directed light output.

FIGS. 2 and 3 are schematics illustrating the implication of the devitrification process at the onset of the process and at the advanced stage of the process, respectively.

FIGS. 4, 5, 6A, 6B illustrate related non-mutually-exclusive embodiments of the invention.

FIGS. 7A, 7B, 7C, and 7D illustrate the use of a disposable version of an embodiment of the invention. FIG. 7B provides the cross-sectional view of the structure of FIG. 7A; FIG. 7C is a detailed view of the working tip of the device of FIG. 7A in a cross-sectional view; and FIG. 7D is an isometric view of the structure of FIG. 7A.

Generally, the sizes and relative scales of elements in Drawings may be set to be different from actual ones to appropriately facilitate simplicity, clarity, and understanding of the Drawings. For the same reason, not all elements present in one Drawing may necessarily be shown in another. While specific embodiments are depicted in the figures with the understanding that the disclosure is intended to be illustrative, these specific embodiments are not intended to limit the scope of invention the implementations of which are described and illustrated herein.

### DETAILED DESCRIPTION

Modifications of optical fiber (usually made by addition to the optical fiber) that are configured to alter the otherwise axially-directed light output (and, for example, produce an output exiting the fiber-optic-based element transversely to the optical axis) typically turn on employing a Total Internal Reflection (TIR) bevel on a fiber end facet. Fiber optic elements based upon an on-fiber bevel polished TIR are limited in the maximum off-axis angle (measured with respect to the fiber longitudinal axis) for efficient redirection of emission by the Numerical Aperture (NA) of the fiber and the actual light mode filling of the fiber (which is defined by laser launch conditions, mode mixing due to fiber length and tortuosity, refractive index of the fiber core at the laser line(s) used, and so on) at the TIR bevel. For example, while the Snell's law might predict a 48° off-axis TIR maximum angle for a light ray propagating in the fiber on and along the fiber longitudinal axis (where the fiber NA is mode-filled at 0.22), one must consider the worst case angle imparting the TIR bevel at the maximum off-fiber-longitudinal-axis angle of about 8.7 degrees (as determined with the use of NA and the glass refractive index) in determining that the actual maximum TIR angle is 39.3° off the longitudinal axis, where the central, lateral output ray is found at 78.6° rather than 96°.

This non-orthogonal output limitation gives rise to an asymmetric distortion of the distribution of radiant intensity and/or irradiance at the output spot at targets that are orthogonal to the fiber's longitudinal axis, with the result that such output spots are elliptical rather than round and radiant intensity is higher near the proximal elliptical vertex rather than the elliptical center. Additional spatial distortions of the light output formed by such “side-fire fiber” result from unintended total internal reflections caused by the complexity of angular and spatial mode fill within multimode, large core optical fibers (such as for example non-medial modes or skew modes), and from complex refraction disclosed by Pon (U.S. Pat. No. 5,428,699) at the fiber cylindrical output surface, refraction at the cylindrical surfaces (whether internal or external) of the protective capsule of the fiber or just refraction at the output surface of the fiber capsule in the case of fused fiber designs (list prior art examples like Griffin's U.S. Pat. No. 5,562,657 and Breke's U.S. Pat. No. 5,537,499), particularly when such fiber optics operates in air or induced steam bubbles.

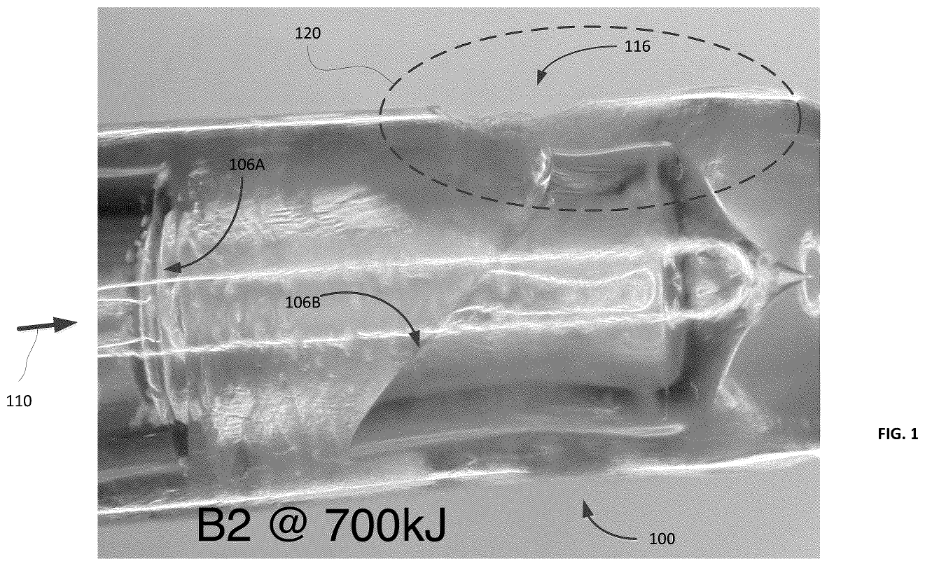

The mechanism of physical change and/or damage of the surface of the optical fiber through which light is delivered may be referred to as a Devitrification Failure Cascade. Indeed, light-caused damage to output surfaces of optical fiber systems (and particularly side-fire fiber systems) appears as devitrification in early stages (see FIG. 1 for an illustration of the damage caused by light delivered through the side surface of a cap with which the side-fire fiber is equipped) and can proceed to extremes where “pits” as deep as millimeters are observed, largely following the highest radiant intensity pathway of the fiber output. While an argument can be envisioned against the damage cascade taking place due to the presence of the optical output (and turning on a position that devitrification of fused quartz or fused silica cannot possibly occur at the temperatures seen in endo-surgery procedures, in which such capped optical fibers are used), such position would be uninformed in that empirical observations show that extreme temperatures and heating of the optical materials are indeed taking place in highly localized microenvironments (particularly in fused silica) and the otherwise relatively low temperatures at which the overall procedure occurs does not negate the presence of such localized microenvironments. Indeed, as experience shows, fused quartz can incandesce in surgery, for example, and metallic components melt, providing temperatures far in excess of those required for devitrification. Devitrification is also promoted by alkali metal ions and counter anions, ions in abundant supply in surgical sites irrigated with saline.

FIG. 1 presents the enlarged view of an optical cap 100 (containing an optical fiber within, delivering light inside the cap 100 in a direction denoted with an arrow 110 towards the internal optical element that is axially bound within the hollow of the cap 100 by surfaces 106A, 106B). Non-limiting examples of similar structures are discussed, for instance, in U.S. Pat. No. 9,323,005 or 11,097,395 (the disclosure of each of which is incorporated herein by reference). Here, as shown, a portion 116 of the output surface of the cap 100 is shown to be structurally-modified by light (damaged) in the area interacting with light that is delivered by the optical fiber and is reflected internally to the cap 100 and is redirected radially outwardly by the surface 106B. Such damaged portion is marked within the ellipse 120.

Even putting aside the arguments about the precise cause of the detrimental change of the structure of the optical material occurring during the operation of the surgical fiber, the process of damage has to be stopped or at least slowed down sufficiently to render the optical-fiber-based system less prone to such damage.

The idea of the invention stems from the realization that not only the devitrification process is the cause of the “frosting” and pitting of glass observed in a side-fire fiber and other types of optical fibers, but that the profile of the light output produced by a side-fire fiber optical system actually affects the initiation of the devitrification process. To this end, it is submitted that in practice of utilization of surgical optical fiber devices, a typically Gaussian profile of the light output (produced substantially in the lowest spatial mode) provides radiant intensity levels at the central portion of the radiation spot more than sufficient for laser ablation, while at the same time the intensity levels at the periphery of the spot of light remain insufficient for vaporizing the biological tissue which this light irradiates during the surgery procedure.

Therefore, the “effective” spot area—that portion of the area irradiated with the light output under which tissue actually ablates—is smaller than the total spot area until the damage of the glass surface in the central portion of the glass area interacting with light output (and corresponding to the central portion of the Gaussian distribution of such light output) progresses to the point when such central portion of the glass area changes its optical and/or structural properties to effectively “flatten” the beam profile by scattering light energy at the peak of the Gaussian light distribution into the lower radiant intensity annulus that surrounds the peak, thereby producing a larger area of surgical efficacy. This enlargement of the area of surgical efficacy is maintained until the damage of the glass surface has progressed as far as to scatter the lower radiant intensity annulus significantly and returning the efficacy area substantially to that initially observed but degrading thereafter.

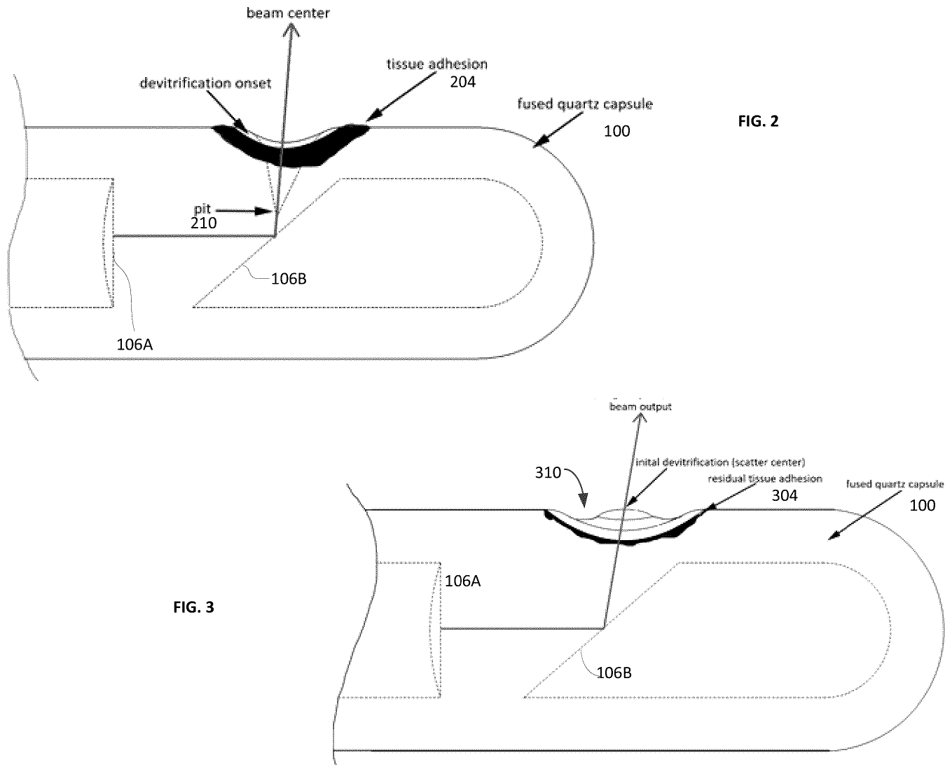

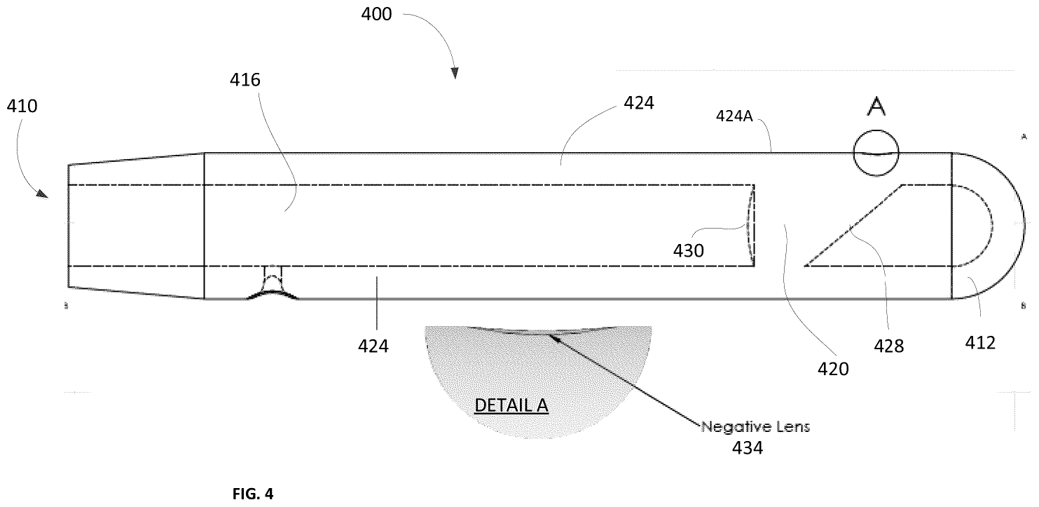

During this process, the following phenomenon can be observed in practice repeatedly—the tissue that encounters/contacts the output surface within such low-intensity peripheral region of the laser light spot simply adheres to the glass and burns, slowly at first but faster as it degrades and absorbs the laser energy increasingly. (See the schematic illustrations of FIGS. 2 and 3.) The glass material adjacent to this “ring of adhesion” (the periphery of the Gaussian spot, so to speak) heats preferentially and the very center of the typically small spot heats the most. It is believed that the hot glass allows for faster intercalation of ions and these ions, in turn, reduce the viscosity of the glass. The two processes eventually reach a point where the glass can assume a lower energy structure because the viscosity that held it back prior to that is no longer present: high cristobalite forms.

As a skilled artisan well knows, laser surgery procedure is far from being continuous and uninterrupted in terms of in application of laser energy to the target; surgeons spend more time examining progress and repositioning the fiber than actually “lasing”. When the laser is off, the fiber delivering radiation to the target cools off and the high cristobalite rearranges to β-cristobalite, a lower energy, crystalline form of silica that has a different density than fused silica. This density difference causes spalling and sloughing of the crystals, which appears as frosted in the initial stages and as a deep pit in extreme cases. Typically, the damage to the fiber output surface causes scattering of the laser spot, creating more areas of tissue adhesion (204, FIG. 2) and the process feeds upon itself. As understood, there is present mild scattering of light that is localized in the center of the beam profile (where laser power is present in excess of the tissue vaporization threshold) into the remainder of the spot profile, which elevates the radiant intensity (that was initially insufficient for vaporization) to a level above the tissue vaporization threshold, removing a portion of the charred tissue ring for some increased radius (see “residual tissue adhesion” 304 in FIG. 3). In this scenario, the effective output spot becomes more surgically effective with minor damage and, in fact, damage could be halted entirely were the radiant profile to become essentially a flat(ter) top or “top hat”, where the transition for vaporization to no effect is essentially instantaneous.

While no fiber has shown to behave in this specific manner precisely, yet, a related scenario has now been consistently observed—for example, in experiments with the Patriot 200W thulium fiber laser output at about 1940 nm. Here, where output laser spots were sufficiently large, the ring of adhesion became sufficiently thin and distant from the center of the spot so that the erosion of the glass material near the center of the output light profile reduced even while devitrification and erosion continued closer to the charred adhesion ring. The adhesion ring is, effectively, pushed outward far enough that the center of the frosted spot on the glass surface is no longer close enough to be the hottest location on the output surface. Now, the thermal conductivity of the fused silica in too low for the heat to concentrate at the center so a ring of erosion (see the ring-like groove 310, FIG. 3, referred to herein as a caldera pattern) forms instead of the usual central pit (210, FIG. 2).

It is noteworthy that the total laser energy at which this characteristic caldera erosion pattern was observed was approximately 10-fold (˜ about one million joules) than that typically producing fatal damage in prior fiber designs (˜ one hundred thousand joules) and that the pattern of erosion was consistent for all “large spot” fibers tested. Further examination of the progression of fiber output properties showed that initial devitrification was limited to the center of the output spot and that the adhesion ring migrated outward as the surface at the center of the output became more scattering: the early damage flattened the output profile, effectively increasing the surgical efficiency by increasing the area of the output that was at or above the vaporization threshold for radiant intensity, pushing the adhesion ring outward and thinning the width of adhesion such that the heat was no longer sufficiently close to the center of the output spot to concentrate at that point, but instead could only reach sufficient temperatures for the rapid intercalation of alkali metal ions within a ring surrounding the area of initial devitrification.

One additional observation from testing the large spot fibers for higher power lasers is germane: tissue vaporization efficiency improved over the first one hundred thousand joules delivered to tissue phantoms (sirloin in this case) and remained higher than the initial efficiency for approximately two hundred thousand additional joules delivered. Devitrification appeared to slow in progression during the period of most efficient tissue vaporization, which is consistent with the devitrification failure cascade thesis. These observations suggests that the original spatial profile of the laser light output from the optical fiber system was indeed altered in the process of operation to a flatter profile by the initial devitrification at the center of the initial output spot, and that the more efficient delivery of laser power to tissue was accompanied by a reduction of laser energy available for tissue adhesion and charring: reduced energy available to heat the output capsule.

It follows that performance of the optical-fiber-based system used in surgery would clearly benefit from altering the output profile of the side fire fiber from Gaussian/Lambertian toward a flatter (if possible—approaching a “top-hat”) profile ahead of time, that is—before applying the device to biological tissue. Were such beam shaping done in a manner that did not seed further degradation, i.e., by a means other than devitrification scatter, it may be possible to reduce tissue adhesion, charring, and capsule heating sufficiently to prevent the initiation of the devitrification failure cascade.

Having advantage of the above discussion, a skilled person now will readily appreciate that the idea of the invention manifests in intentional production or formation of the initial scattering effect at the optical fiber structure without and prior to waiting for the damage to be naturally started during the use of the structure. In at least some implementations, such scattering effect is achieved by devising an efficient scattering element on the surface of the optical structure or by means of flat top profile inducing optics at various surfaces available for diffractive correction of the spatial profile of the light output. It is believed that by equipping the optical-fiber-based structure with such element(s) it may be possible to prevent the initiation of glass devitrification process completely.

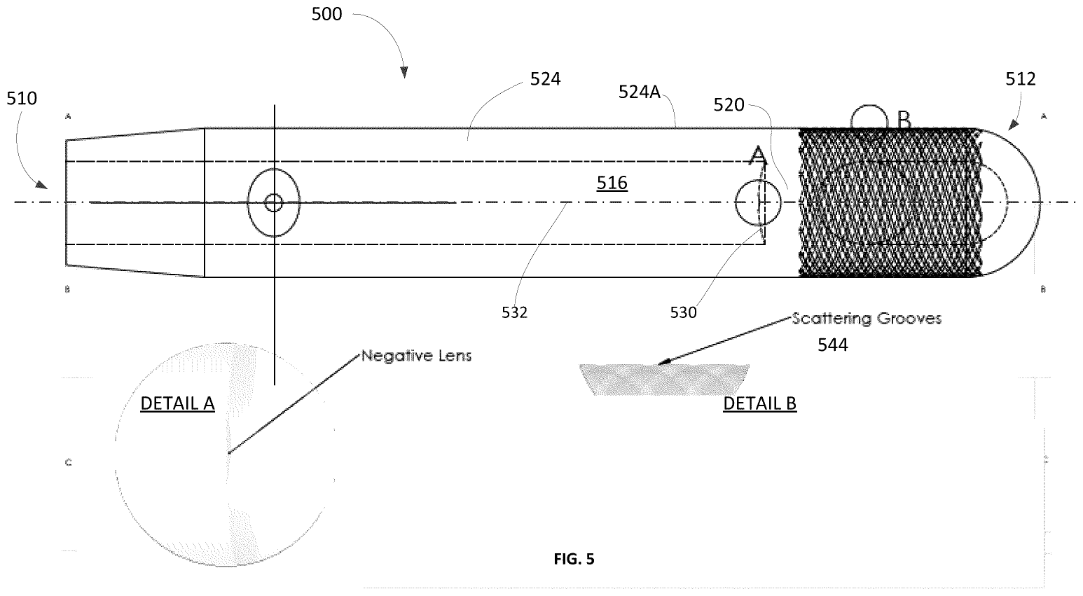

To this end, FIG. 4 presents a schematic of an embodiment of an article of manufacture comprising an optical fiber termination element 400 (which may interchangeably be referred to as a cap or as an optical fiber termination). The cap 400 is implemented as a tubular element having a one-piece construction of an optically-transparent material (for example, fused quartz and/or fused silica), the open input end 410, and the closed or sealed end 412. Access to the hollow 416 of the cap 400 is available through the open end 410, dimensioned to accept an optical fiber (not shown for simplicity of illustration). While for the purposes of describing the implementation of the idea of the invention details of the internal structure of the cap 400 may be substantially irrelevant, in this specific implementation the cap 400 is shown to include a light-guiding section 420 of the cap, which may be fabricated to be monolithic with the substantially-cylindrical wall 424, and which is terminated with the reflecting surface 428. The reflecting surface 428 is configured to redirect optical radiation arriving to it from the open end 410 towards and through the inner and/or outer surfaces of the wall 424 of the cap 400 (substantially in the area of the wall corresponding to mark A). The opposite end of the light-guiding section 420 is terminated with a curved surface 430 configured as a surface of the lens changing the degree or convergence or divergence of light arriving at the lens 430 from the open end 420 upon propagation of this light towards the surface 428. A portion of the hollow or bore 416, then, is terminated at the surface 430. (Generally, however, the internal structure of the cap of an embodiment of the invention can be configured according to each of a variety of options discussed, for example, in U.S. patent application Ser. No. 17/224,354 the disclosure of which is incorporated herein by reference.)

As shown in inset “DETAIL A” of FIG. 4, the outer surface 424A of the wall 424 of the cap 400 is judiciously modified to be inwardly curved—that is, to contain a concavely-shaped relief structure forming an indentation to define a surface of a lens 434 with negative optical power. The so-structured lens, in operation of the article 400, acts to spatially disperse the radiative energy transferred through the area A of the wall 424 to reduce the level of radiative intensity in the central portion of the area as compared to the case when the cap 400 does not contain such a lens 430.

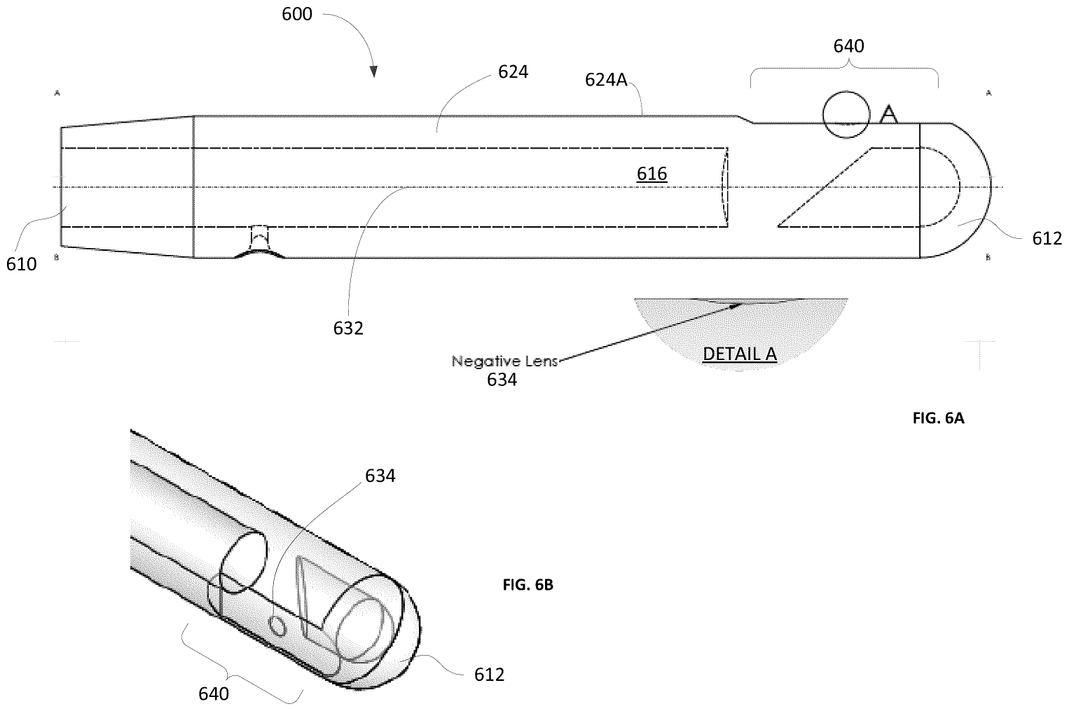

FIG. 5 illustrates a related embodiment 500 of the optical fiber cap, which is similar in structure to the embodiment 400 but which includes at least one of the two modifications detailed in insets A and B. Here, the generally convex curved surface of the light-guiding section 520 of the cap (that separates the open end 510 from the sealed end 512 of the cap 500) is judiciously structured to additionally include an axial indentation, in the central portion of the surface 530. As a result, the surface 530 is configured to be substantially bi-curved: the axial, central portion is concave, thereby providing for a surface of the local lens element with a negative optical power, while the peripheral area of the surface 530 that circumscribes this central portion remains convex. As shown in inset “DETAIL B”, the portion of the outer surface of the wall 524—through which radiation is redirected from inside the cap 500 (when the optical fiber, affixed in the hollow 516 of the cap 500, delivers such radiation towards the surface 530)—may be structured to carry a surface relief thereon. Such surface relief structure 544 (marked as “scattering grooves” in inset B) is judiciously configured to have a beam of light transmitted therethrough while changing a degree of divergence (or convergence) of such a beam. The surface relief structure may include one or more of a periodic surface corrugation pattern and an aperiodic surface corrugation pattern and, at least in one specific implementation, be dimensioned to substantially circumscribe the cap 520 around the axis 532. The structure 544 may include an optical metasurface (that is, a patterned layer with features dimensioned to be smaller than the operational wavelength of the light used during the exploitation of the cap 500, sub-wavelength; not shown in Figures for simplicity of illustration).

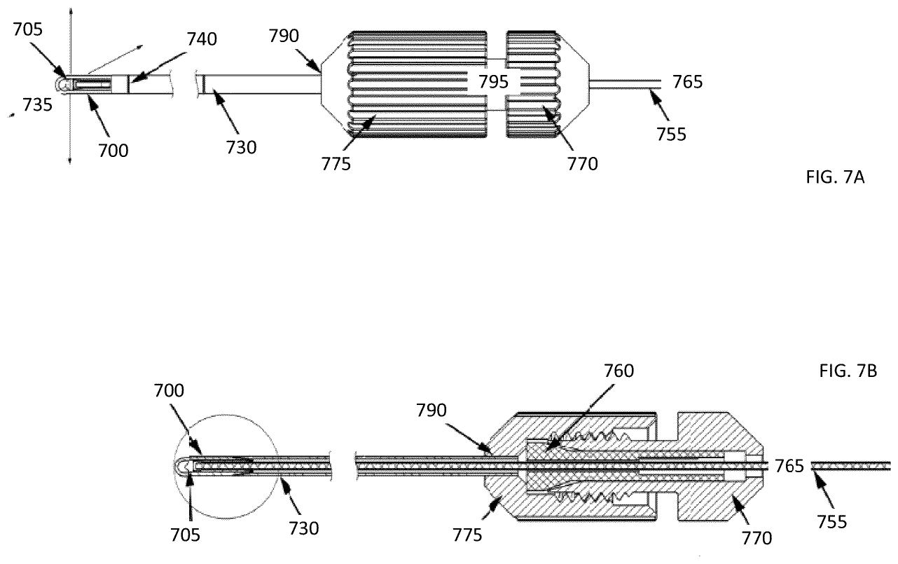

FIGS. 6A, 6B illustrate yet another related and non-limiting embodiment 600 of the invention, which represents a modification of the embodiment 400 of FIG. 4. FIG. 6A includes inset “DETAIL A” depicting a relief structure of the outer surface 624A of the wall 624 of the embodiment 600, while FIG. 6B shows schematically a portion of the embodiment in perspective view. Here, in comparison with the structure of the embodiment 400, the relief structure 634 configured as a concave surface is formed on a substantially planar facet area formed along the axis 632 of the cap 600. The hollow is indicated with the numeral 616.

As noted in U.S. patent application Ser. No. 17/224, the above-discussed article of manufacture 400, 500, 600 may be used as part of a larger optical-fiber-based system configured for surgical applications.

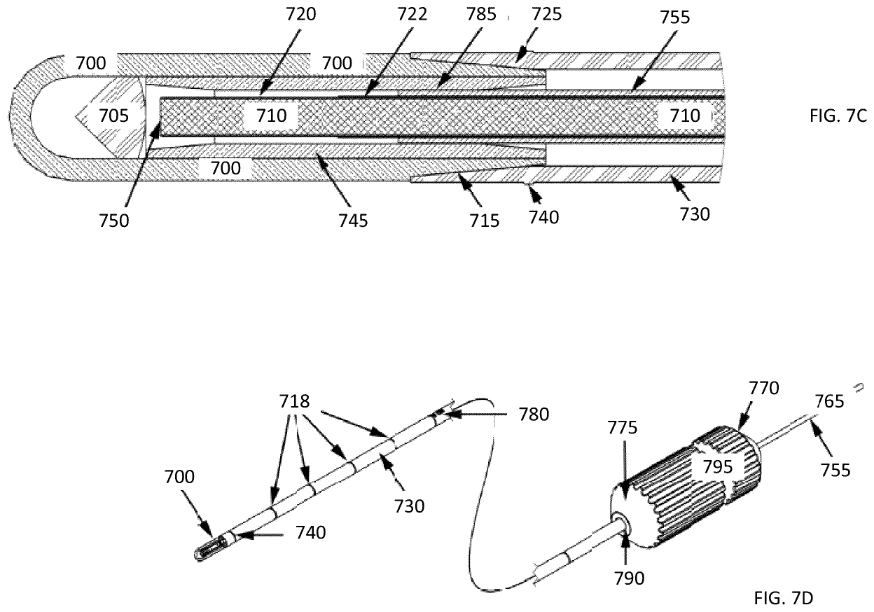

For example, FIGS. 7A, 7B, 7C, and 7D depict a resposable embodiment of the invention, where the term “resposable” addresses a device within which a component or components, such as a surgical tip or patient contact assembly, is/are optionally disposable and in which one or more other components, such as a transmitting fiber optic conduit for use with the optionally disposable part, is reusable. A transmitting optical fiber 765, which may be a polyamide or polyamide-imide (e.g. nylon) buffered 755, fluoropolymer coated 722, fluorine-doped silica clad 720 and silica core optical fiber 710, has a prepared output tip 750 that is protected by a centering sleeve 745 made of glass, ceramic or metal, disposed about the fiber outer diameter and attached 785 with adhesive or crimping to the fiber buffer 755. Other mechanisms for protecting the transmitting fiber tip 750 will be apparent to those skilled in the art. Preferably, the fiber and centering sleeve are not attached to the fiber cap 700 (configured to implement the idea of the invention as discussed above in reference to at least one of FIGS. 4, 5, 6A, 6B) that contains the radial emission optical element 705. Notably, in the example of FIGS. 7A, 7B, 7C, 7D the internal structure of the cap 700 is different from that of the embodiments 400, 500, and 600. In one instance, the fiber cap is chamfered 715 to mate with a matching chamfer 725 within a cannula 730, preferably a semi-rigid cannula. In another instance, the fiber cap 700 is hermetically attached (e.g., adhesively) to the cannula 730.

The semi-rigid cannula 730 can be attached 790 by means of adhesive, solvent welding or other method to a cannula-mount segment 775 of a fiber control device (e.g., a pin vise) 795 having components made of rigid polymer or metal. Accordingly, the fiber cap 700, cannula 730 and cannula-mount segment 775 form a detachable subassembly that includes the entirety of patient contacting components. Notably, the fiber control device 795 includes at least two separable components: a cannula-mount segment 775 and a fiber-holding segment 770. In one instance, the cannula-mount segment 775 and the fiber-holding segment 770 are reversibly affixed by, for example, matching screw threading. Additional reversibly means of affixing the cannula-mount segment 775 and the fiber-holding segment 770 include snap closures, pin-vise connections, a bayonet mount, a BNC-style connector, a RF connector, a UHF connector, a SMA connector, a SMB connector, a SMC connector, a TNC connector, a N connector, a C connector, or the like. The laser connector (not depicted), transmitting fiber optic conduit 765 and the fiber-holding segment 770 (which can include a fiber retaining collet 760) represent a second subassembly comprised of components that are not in patient contact and represent approximately 80% of the device cost.

As a placement aid to use in surgery, the cannula 730 may be marked with clearly visible bands spaced one centimeter apart 718, where the first mark 740 is positioned one centimeter proximal to the radial output 735 indicated by the small arrows. Additional markings 780 provide a guide to the depth of insertion; in this case the marking 780 reads “5 cm”. By loosening the fiber control device 795, the cap 700, cannula 730 and the cannula-mount segment 775 may be discarded and replaced intraoperatively, greatly reducing the cost of disposable material.

In one related embodiment, the control device 795 can be alternatively structured as that discussed in a US Design Patent Application No. 29/853,120 and/or U.S. patent application Ser. No. 18/228,983, the disclosure of each of which is incorporated herein by reference.

It is understood that, in addition, the an optical fiber element with which an embodiment of the optical fiber cap disclosed herein can be used in practice can be formatted to contain at least a) an output end including a fiber taper and/or an output facet structured as a lenslet (thereby changing a degree of spatial diversion of light at the output of the fiber) and/or b) an fiber end termination structured according to the idea discussed in reference to FIGS. 7 and/or 8 and/or 9 of the U.S. patent application Ser. No. 17/847,608 now published as US 2023/0023074, the disclosure of which is incorporated herein by reference.

Overall, understandably, minimization of such spatial distortions of the laser light output that occur during the surgical procedure performed with the use of, for example, a side-fire optical fiber contraption remains a need in related art. The rationale behind the efforts to minimize such spatial distortions, which lead to the current invention, is multifold: to improve surgical efficiency where the fiber output radiant intensity mimics the spatial uniformity of an axial output fiber and, perhaps more critically, to reduce the unintended effects on the biological tissue in the spatial regions or zones of the light output where radiant intensity level is sub-therapeutic (that is, areas of the fiber-optic protective capsule where laser radiant intensity is below the required therapeutic level) as well as to reduce tissue adhesion to the fiber optics within such regions or zones.

Surgical practice proves that—while small, spatially-concentrated output spots of laser light may be favored in theory—are not desirable for surgical efficiency. Surgical efficiency, particularly where surgical lasers offer higher and higher output power, is optimized where the maximum output spot area is at or above the radiant intensity required for vaporization of tissue (aka the vaporization threshold), and where radiant intensities within that area do not greatly exceed the vaporization threshold: higher laser powers require larger output spots in order to take advantage of the higher powers that are available. A spatial profile of the laser light output delivered by a surgical fiber that is substantially flat (as close to a “top hat” profile as possible) where the radiant intensity is above the vaporization threshold theoretically represents the highest surgical efficiency and, were it achievable, such a spot where the radiant intensity produced a spot “edge” would also offer a minimal area that supported tissue adhesion.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. In one specific case, the terms “approximately”, “substantially”, and “about”, when used in reference to a numerical value, represent a range of plus or minus 20% with respect to the specified value, more preferably plus or minus 10%, even more preferably plus or minus 5%, most preferably plus or minus 2% with respect to the specified value. As a non-limiting example, two values being “substantially equal” to one another implies that the difference between the two values may be within the range of +/−20% of the value itself, preferably within the +/−10% range of the value itself, more preferably within the range of +/−5% of the value itself, and even more preferably within the range of +/−2% or less of the value itself. The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes.

The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes.

While the invention is described through the above-described exemplary embodiments, it will be understood by those of ordinary skill in the art that modifications to, and variations of, the illustrated embodiments may be made without departing from the inventive concepts disclosed herein. Disclosed aspects, or portions of these aspects, may be combined in ways not listed above. Accordingly, the invention should not be viewed as being limited to the disclosed embodiment(s).

1. An article of manufacture comprising:
an optical fiber termination cap that includes:
an optically-transparent tube having a tube axis, an open end dimensioned to receive an optical fiber along a tube axis, and a closed end;
an optical element that has an output optical element surface transverse to the tube axis and that is configured inside the tube
(i) to receive light along the tube axis from the open end and,
(ii) upon interaction of said light with the output optical element surface, to form a first beam of light having a first degree of spatial divergence and directed transversely to the tube axis along an output axis through a wall of the tube;
wherein the wall of the tube contains a surface relief structure intersecting said output axis, the surface relief structure being configured to transmit the first beam of light therethrough while changing the first degree of spatial divergence to a different second degree of spatial divergence.

2. An article of manufacture according to claim 1, wherein the surface relief structure includes one or more of a periodic surface corrugation pattern and an aperiodic surface corrugation pattern.

3. An article of manufacture according to claim 1, wherein the surface relief structure is dimensioned to substantially circumscribe the tube around the tube axis.

4. An article of manufacture according to claim 1, wherein the surface relief structure is formed at least in part on a substantially-planar portion of an outer surface of the wall of the tube.

5. An article of manufacture according to claim 1, wherein the surface relief structure is dimensioned to form a lens element.

6. An article of manufacture according to claim 1, wherein said output optical element surface is substantially planar and/or whereon the optical element includes an input optical element surface defining a surface of a lens to receive said light along the tube axis from the open end and configured to form an intermediate beam of light that has the first degree of spatial divergence and that propagates towards the output optical element surface.

7. An article of manufacture according to claim 6, wherein, when the optical element includes an input optical element surface, said input optical element surface is configured to direct the intermediate beam of light through a body of the optical element.

8. An article of manufacture according to claim 1, wherein the surface relief structure includes an optical metasurface.

9. An article of manufacture according to one claim 1, further comprising a cannula connected to said cap.

10. An article of manufacture according to claim 9, comprising a cannula-mount segment of a fiber-control device affixed to the cannula.

11. An article of manufacture according to claim 10, wherein the open end is dimensioned to receive an optical fiber along a tube axis, and further comprising the optical fiber cooperated with the fiber control device and inserted into the optical fiber termination cap.

12. An article of manufacture according to claim 11, further comprising a centering sleeve disposed about an output tip of the optical fiber.

13. An article of manufacture according to claim 1, wherein the open end is dimensioned to receive an optical fiber along a tube axis, and further comprising an optical fiber inserted into the tube.

14. An article of manufacture according to claim 13, wherein an output facet of the optical fiber is shaped to include a curved surface.

15. A method comprising:
interacting light, propagating axially within an optical termination cap from an open end of an optical termination cap towards a closed end of the optical termination cap, with an optical body that is located inside the optical termination cap and that has an optical body output surface transverse to an optical termination cap axis, wherein said optical termination cap includes an optically-transparent tube having said open end, said closed end, and said optical termination cap axis;
upon so interacting, forming a first beam of light having a first degree of spatial divergence and directed transversely to the optical termination cap axis and along an output axis through a wall of the tube; and
transforming the first beam of light into a second beam of light having a second degree of spatial divergence by transmitting the first beam of light through a surface relief structure on the wall of the tube, the second degree of spatial divergence being different from the first degree of spatial divergence.

16. A method according to claim 15, wherein the open end is dimensioned to receive an optical fiber along the optical termination cap axis, and further comprising outcoupling said light through an output facet of an optical fiber secured in the optical fiber termination cap.

17. A method according to claim 16,
(17A) wherein said outcoupling light includes outcoupling said light from an output facet of an optical fiber, and/or
(17B) wherein said outcoupling light includes outcoupling said light from the output facet of the optical fiber, said output facet being a facet of the optical fiber taper, and/or
(17C) wherein said outcoupling light includes outcoupling said light from the output face, said output facet being a facet of the optical fiber element that includes:
an optical fiber having a fiber core and a fiber cladding, and
an optical termination element in contact with an input facet of the optical fiber, the optical termination element having a front surface, a termination core, and a termination cladding,
wherein a first ratio of a termination core diameter to a termination cladding diameter is substantially equal to a second ratio of a fiber core diameter to the fiber cladding diameter.

18. A method according to claim 17, wherein said output facet of the optical fiber includes a curved surface transverse to the optical termination cap axis.

19. A method according to claim 15, wherein said transforming the first beam includes transmitting light of the first beam through the surface relief structure that includes one or more of a periodic surface corrugation pattern and an aperiodic surface corrugation pattern and/or that is dimensioned to form a lens element.

20. A method according to claim 15, wherein said transforming includes transmitting the first beam through the surface relief structure that is dimensioned to substantially circumscribe the tube around the optical termination cap axis and/or that is formed at least in part on a substantially-planar portion of an outer surface of the wall of the tube and/or that includes an optical metasurface.

21. A method according to claim 15, further comprising, when the optical termination cap contains an optical fiber inserted into the cap through the open end and spatially secured with respect to the cap, operating a fiber control device cooperated with said optical fiber to spatially reposition the optical termination cap axis.
